(19) United States
(12) Patent Application Publication
(10) Pub. No.: US 2005/0027302 A1
(43) Pub. Date: Feb. 3, 2005

(54) Resilient thimble for ball head of prosthetic joint

(75) Inventors: Christophe Cueille, Missy (FR); Patrick Raugel, Charge (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 10/926,643

(22) Filed: Aug. 26, 2004

Related U.S. Application Data
(62) Division of application No. 10/243,415, filed on Sep. 13, 2002.

(30) Foreign Application Priority Data
Sep. 14, 2001 (GB) 0122295.9

(51) Int. Cl. A61B 17/92; A61F 2/36

(52) U.S. Cl. 606/99; 623/22.45

(57) ABSTRACT

A head component for use in a prosthetic joint such as a hip joint. The head has a body adapted to engage a natural acetabulum or a prosthetic acetabular cup component. The head has a recess with a resilient sleeve located therein adapted to engage the trunnion of a prosthetic stem component with which it is to be used. The sleeve material is more deformable than the trunnion material and can deform under sufficient force to absorb any unevenness in the trunnion surface.

### CROSS-REFERENCE TO RELATED APPLICATION

This application is a divisional of U.S. application Ser. No. 10/243,425 filed Sep. 13, 2002.

### BACKGROUND OF THE INVENTION

This invention relates to the ball head of a prosthetic joint particularly, but not exclusively, for use in a replacement hip joint, a prosthesis incorporating such a ball head, and a method of assembly thereof.

For example, the Exeter and Charnley type femoral prostheses are well known and comprise a stem for implantation into the medullary canal and have a neck at the proximal end which carries a ball or spherical head portion or a spigot or trunnion for cooperation with a ball head component. With the Exeter design the ball head is dimensioned to cooperate with an acetabular cup prosthesis implanted into the acetabulum. With the Charnley design the ball head is sometimes dimensioned to cooperate with the natural acetabulum.

It is common for the femoral prostheses to be provided with a modular head rather than an integral ball head portion, because the stem can be more readily implanted into a medullary canal without the ball head attached. Further, if revision surgery is required to correct a failing spherical head it can be removed from the spigot or trunnion and replaced without removing the stem from the medullary canal. This reduction in surgery is beneficial for patients, particularly the elderly.

It is also known for ball heads to be constructed from a ceramics material as it provides a particularly suitable bearing surface. However, ball heads of this type are relatively brittle and can be damaged when subjected to loading of the joint by the patient after surgery, for example during walking. In these circumstances the spherical head needs to be replaced in revision surgery, which is bad for patients, particularly the elderly.

It is further known for the head spigots or trunnions of femoral stem components to suffer damage in the form of scratches. This can occur during fitting or removal of the ball head and during normal loading of the joint if the ball head is not securely fitted to the head spigot and makes minute movements thereon. It can also occur as the result of the ball head failure. Unfortunately, a new ceramics ball head cannot be fitted onto a damaged head spigot. Scratches on the surface of a head spigot create ridges in the recess receiving the head which can fracture a ceramics ball head during fitting or joint loading, leading to prosthesis failure. If it is found during revision surgery that the head spigot has also suffered damage for whatever reason, the femoral stem component has to be replaced. This significantly extends surgery time, which can be harmful to patients, particularly the elderly.

### SUMMARY OF THE INVENTION

Therefore, it is proposed that by manufacturing a ceramic ball head with a relatively resilient and deformable thimble or sleeve located therein, some of these problems can be overcome. In the preferred embodiment, the thimble or sleeve is integrally attached to the head such as by being press-fit therein.

A thimble component made of a relatively resilient and deformable material such as titanium or titanium alloy can perform two functions. Firstly, it can absorb the defects of a damaged head spigot, so an imperfect femoral stem component would not need to be replaced during revision surgery.

Further, the resilient properties of the thimble can help to prevent structural damage being caused to the ceramics ball head by the less resilient head spigot during fitting and removal of the ball head and during loading of the joint. In addition, the thimble or sleeve can help to prevent damage being caused to the head trunnion if the ball head is not securely fitted thereon and makes minute movements during loading.

Therefore, according to the present invention a head component for use in a prosthetic joint comprises a body adapted to engage a natural or a prosthetic cup component and a resilient thimble located therein adapted to engage the head spigot of a prosthetic stem component with which it is to be used.

The invention includes a method for forming and attaching a part spherical head of a prosthetic femoral component to a damaged trunnion extending from a bone contacting part of the femoral component. The method includes forming a head having a recess from a ceramic material; inserting a metal sleeve having a first deformability into the recess with sufficient pressure to integrally couple the sleeve to the head. The head and sleeve assembly is then placed on the metal trunnion having a second deformability. The metal trunnion has an uneven surface with the first deformability being greater than the second. A sufficient force is applied to the head to deform the metal of the sleeve to absorb the unevenness of the surface of the trunnion.

The invention also includes a prosthesis comprising a stem and a neck carrying a head spigot which is engaged with a ball head which is adapted to engage said head spigot. The ball or spherical head component being adapted to engage a natural or a prosthetic cup component.

### BRIEF DESCRIPTION OF THE DRAWINGS

The invention can be performed in various ways but one embodiment will now be described by way of example and with reference to the accompanying drawings in which:

FIG. 1 is a cross-sectional side view of a spherical head according to the present invention;

FIG. 2 is a cross-sectional top view of a prior art ceramics spherical head fitted to a damaged head trunnion;

FIG. 3 is a cross-sectional top view of a ceramics spherical head according to the present invention fitted to a damaged head trunnion;

FIG. 4 is a side view of the spherical head in FIG. 1 being fitted onto the trunnion of a femoral prosthesis located in the medullary canal, with the spherical head and the femoral prosthesis shown in cross-section; and

FIG. 5 is a cross-sectional side view of a total hip prosthesis according to the present invention.

### DETAILED DESCRIPTION

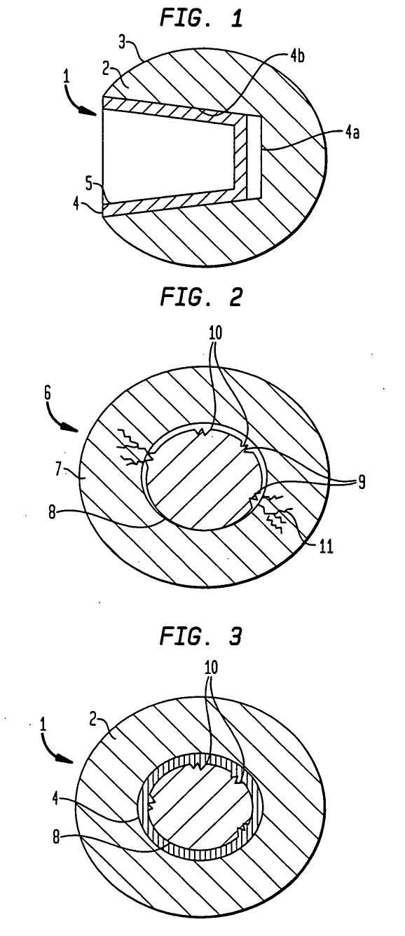

As shown in FIG. 1 a part-spherical or ball head 1 for a femoral prosthesis which head 1 has a body 2 formed from a ceramics material, for example, alumina. The body 2 has a smooth bearing surface 3. The body includes a recess 4b which, in the preferred embodiment, is conically tapered. The recess 4b could also be cylindrical. The ball head 1 is provided with a thimble or sleeve 4 constructed from a relatively resilient and deformable metal material when compared to the deformability of other metals such as stainless steel and cobalt-chrome molybdenum alloy (Vitallium®), for example titanium. In one embodiment, the sleeve 4 is secured inside the body 2 by means of a preloading procedure such as press fitting during construction. A void 4a is left between the sleeve 4 and the body 2. In the preferred embodiment, the inner surface 5 of the thimble 4 is formed as a Morse tapered socket or recess. The outer body contacting surface of the sleeve preferably is shaped to match the shape of the recess 4b. The sleeve 4 could also be captured within the recess of the spherical head during the manufacturing process of the head.

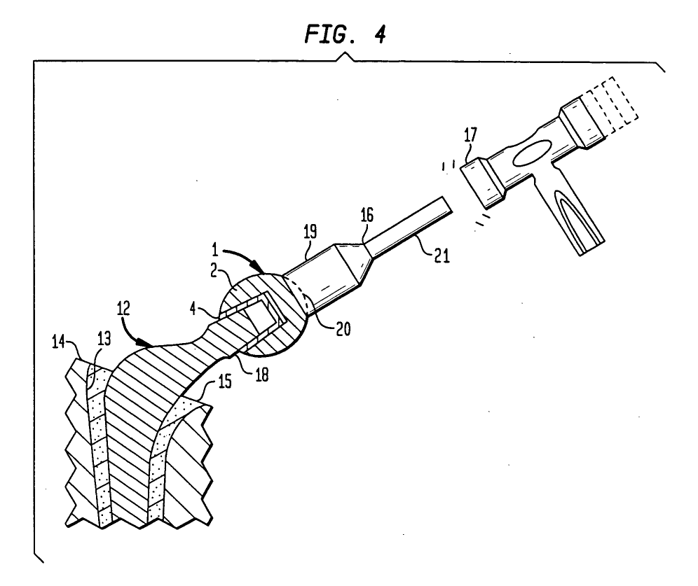

In the preferred embodiment, the ball head 1 and the resilient thimble 4 can be assembled together prior to assembly to a femoral stem component 12 (FIG. 4). The head 1 and thimble 4 are preloaded under a force between 400N and 800N. This force is higher than any force generated during use or during removal tending to separate the sleeve 4 from the body recess 4b of the head.

As shown in FIG. 2, a prior art ball head 6 has a body 7 formed from a ceramics material, but it is not provided with an inner sleeve. The ball head 6 has been fitted into a head spigot 8 which may be made of stainless steel or Vitallium® alloy, which has suffered damage in the form of vertical scratches 9 (shown here in exaggerated size). The material displaced by the scratches 9 has formed ridges 10 rendering the trunnion surface uneven. The ridges 10 prevent the ball head 6 from making full contact with the head trunnion or spigot 8. As a result of the partial contact the body 7 is subjected to extreme-stress during loading. The leads to fractures 11, and subsequent ceramic ball head failure.

FIG. 3 shows the ball head 1 as shown in FIG. 1, fitted onto the damaged head spigot 8 as shown in FIG. 2. The titanium or titanium-alloy sleeve 4 has absorbed the shape of the ridges 10, and made full contact with the head spigot 8. As a result of the deformation of the softer thimble 4 the body 2 is subjected to equal stresses during loading and no fractures occur.

In FIG. 4 ball head 1 is in the process of being attached to femoral stem component 12 which is already implanted in the medullary canal 13 of the femur 14 by means of cement 15. (A collarless Exeter type femoral stem component is shown here, but it will be appreciated that any known type of femoral stem component which features a head spigot or trunnion can be used). Reduction handle 16 and surgical hammer 17 are used to secure the ball head 1 to the undamaged head spigot 18 of the femoral stem component 12. The reduction handle 16 has a head section 19 which is made from a plastics material, and features a hemispherical impaction end 20 for cooperation with the ball head 1, and a body section 21 adapted to cooperate with the surgical hammer 17. The thimble 4 acts as a resilient damping means between the body of the ball head 2 and the head spigot 18 when impact pressure is applied to the body of the ball head 2 via the hammer 17 and the reduction handle 16, which helps to prevent ball head failure during surgery.

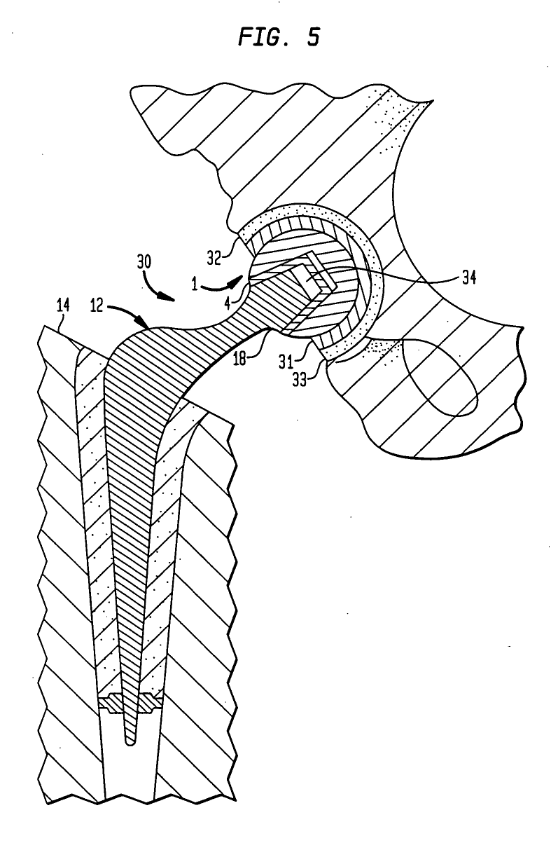

In FIG. 5 complete hip prosthesis 30 comprises a ball head 1 fully secured to a femoral stem component 12, and an acetabular cup component 31 which is implanted into the acetabulum 32 by means of cement 33. Void 34 is left between the head spigot 18 and the thimble 4. During normal gait the ball head 1 is subjected to the known multiple loads which can cause fractures to traditional ball heads. However, sleeve 4 acts as a resilient dampening means between the femur 14 and the acetabulum 32 during normal gait and prevents the multiple loads resulting in ball head failure.

Thus, this invention reduces the chances of ball head failure due to a damaged head spigot during revision surgery and during loading of the joint thanks to the deformable properties of the thimble. This prevents the need to replace prostheses which are found during revision surgery to have damaged or scratched head spigots or trunnion. Moreover, the internal dimensions of the thimble can be varied as required for any particular head spigot. Thus, for the same size of ball head the internal taper, length or diameter of the thimble can be varied.

Further, this invention provides a complete prosthesis comprising a stem portion provided with a head spigot, which is connected to a ball head by means of a resilient thimble. Such a prosthesis is less likely to suffer failure during loading of the joint because of the resilient properties of the thimble.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for forming a part-spherical head of a prosthetic femoral component and attaching it to a damaged metal trunnion of a bone contacting part of the femoral component comprising:
forming a part spherical head having a recess from a ceramic material;
inserting a metal sleeve having a first deformability into the recess with sufficient pressure to integrally couple the sleeve to the head;
placing the head and sleeve assembly on the metal trunnion on the bone contacting part, the trunnion having a second deformability, the trunnion having an uneven surface and wherein the first deformability is greater than the second; and
applying sufficient force to said head to deform the metal of the sleeve and absorb the unevenness of the surface of the trunnion.

2. The method as set forth in claim 1, wherein a void is left between the top of the sleeve and the base of the recess provided in the body.

3. The method as set forth in claim 2, wherein the recess is conically tapered inwardly.

4. The method as set forth in claim 3 has an outer surface which is conically tapered to match the recess.

5. The method as set forth in claim 4, wherein the sleeve is constructed from titanium or a titanium alloy.

6. The method as set forth in claim 5 wherein the metal trunnion is made from a cobalt chrome alloy or stainless steel.

7. The method as set forth in claim 1, wherein the sleeve is provided with a Morse tapered inner surface adapted to engage a conical trunnion of a prosthetic stem component with which it is to be used.

8. The method as set forth in claim 1 wherein the pressure applied to integrally couple the sleeve and the head is between 400 N and 800 N.

9. The method as set forth in claim 8 wherein the pressure is applied by press-fitting.

10. The method as set forth in claim 1 wherein the ceramic is alumina.

11. A method for forming a modular head for a metal joint prosthesis comprising:
forming a part-spherical body from a ceramic material;
forming a recess having an open end in said body;
lining said recess with a metal sleeve made of a metal having a deformability greater than the deformability of a metal of said metal joint prosthesis.

12. The method as set forth in claim 11 wherein the ceramic material is alumina.

13. The method as set forth in claim 11 wherein a void is left between a top of the metal sleeve and a base of the recess provided in the body.

14. The method as set forth in claim 11 wherein the recess is conically tapered inwardly from said open end towards said base.

15. The method as set forth in claim 14, said sleeve has an outer surface which is conically tapered to match the recess.

16. The method as set forth in claim 11 wherein the metal of the sleeve is from titanium or a titanium alloy.

17. The method as set forth in claim 16 wherein the metal of the first prosthesis is a cobalt chrome alloy or a stainless steel.

18. The method as set forth in claim 11 wherein the sleeve is provided with a Morse tapered inner surface adapted to engage a conical trunnion of a prosthetic stem component with which it is to be used.

19. The method as set forth in claim 11 wherein said recess is lined by said metal sleeve by applying a preload pressure between said metal sleeve and said recess.

20. The method as set forth in claim 19 wherein the pressure applied to integrally couple the sleeve and the head is between 400 N and 800 N.

21. The method as set forth in claim 20 wherein the pressure is applied by press-fitting.
